(19) United States
(12) Patent Application Publication
(10) Pub. No.: US 2009/0287203 A1
(43) Pub. Date: Nov. 19, 2009

(54) Apparatus and Methods for Retracting an Ablation Balloon

(75) Inventors: James Mazzone, San Jose, CA (US); Steven T. Onishi, Cupertino, CA (US); Byron L. Chun, Castro Valley, CA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC, Maple Grove, MN (US)

(21) Appl. No.: 12/463,842

(22) Filed: May 11, 2009

Related U.S. Application Data
(60) Provisional application No. 61/052,979, filed on May 13, 2008.

(51) Int. Cl. A61B 18/02; A61M 25/10

(52) U.S. Cl. 606/21; 604/96.01

(57) ABSTRACT

Apparatus and methods for retracting a balloon back into a delivery sheath following a medical procedure using the balloon. A handle on the device includes a balloon folding mechanism coupled to a proximal end of a guide wire shaft so that actuating the balloon folding mechanism causes rotation of the guide wire shaft within a catheter body. A distal end of the balloon is attached to a distal end of the guide wire shaft, which extends beyond the distal end of the catheter body, and a proximal end of the balloon is attached to the distal end of the catheter body so that actuating the folding mechanism causes the balloon to wrap around the guide wire shaft. The folding mechanism may also be configured for elongating the balloon. Rotating, or rotating and elongating, the deflated balloon results in a balloon with a smaller profile that may easily be retracted into a delivery sheath.

### RELATED APPLICATION DATA

The present application claims the benefit under 35 U.S.C. §119 to U.S. Provisional Patent Application Ser. No. 61/052,979, filed May 13, 2008. The foregoing application is hereby incorporated by reference into the present application in its entirety.

### FIELD OF THE INVENTION

The present inventions relate to apparatus and methods for retracting an ablation balloon within the distal end of a sheath.

### BACKGROUND

Atrial fibrillation is a condition in which the upper chambers of the heart beat rapidly and irregularly. The current standard of care for treating atrial fibrillation is to administer drugs in order to maintain normal sinus rhythm and/or to decrease ventricular rhythm. Drug treatments, however, may not be sufficiently effective or tolerated by AF patients, warranting additional measures such as cardiac tissue ablation to mitigate the arrhythmia.

Known ablation procedures for treating atrial fibrillation include performing catheter ablation to electrically isolate (i.e. disconnect) the pulmonary veins from the left atrium (LA), create linear lesions (lines of block) in the LA, and target sites of complex fractionation-using radio frequency (RF) energy. Transmural ablation involves ablating cardiac tissue, thereby forming lesions to eliminate the triggers for AF and to break up circuits believed to maintain atrial fibrillation. Such transmural ablation procedures may be endocardial, i.e., performed from inside the atria or ventricles accessed through the veins or arteries of the patient, or epicardial, i.e., performed in the pericardial space through the external surface of the heart using devices introduced through ports in the patient's chest.

While RF transmural ablation has been used effectively in the past, cryogenic ablation has received increased attention for treatment of atrial fibrillation because of the safety benefits of this energy source. Such safety benefits include reduced risk of thrombus/char, reduced risk of damage to collateral structures such as the esophagus, reduced risk of PV stenosis, and the like. One known endocardial cryo-ablation procedure involves inserting a point cryo-ablation catheter through a delivery sheath and into the heart, e.g., delivered percutaneously through the leg of the patient into the femoral vein. Once properly positioned, the tip of the catheter is cooled to a sufficiently low temperature by use of a liquid coolant or refrigerant such as nitrous oxide, e.g., to sub-zero temperatures of about −75° C., in order to freeze tissue believed to conduct signals that cause atrial fibrillation. The frozen tissue eventually dies so that the ablated tissue no longer conducts electrical impulses that are believed to cause or conduct atrial fibrillation signals.

Certain known endocardial cryo-ablation devices include expandable balloons, which are inflated with the liquid coolant or refrigerant. After the ablation is performed, and before the device is withdrawn from the patient, the balloon may be deflated and retracted into the delivery sheath. However, after inflating the balloon, performing the ablation procedure, and deflating the balloon, a user may encounter difficulties in retracting the deflated balloon into the sheath due to the balloon having a profile that is too large to re-enter the sheath. In particular, prior to inflation, the balloon profile is at its smallest, but after inflation, the balloon may deflate into an unpredictable profile and may bunch up at the tip of the sheath during attempts to retract the balloon into the sheath. Thus, increased force is required to retract the deflated balloon, thereby potentially damaging the balloon during the retraction procedure.

### SUMMARY OF THE INVENTION

In one embodiment of the inventions disclosed herein, a tissue ablation system includes a catheter. An elongated member (e.g., a guide wire shaft defining its own lumen) extends through the catheter, with a distal end of the elongated member extending out of a distal end opening of the catheter. An expandable balloon, e.g., a cryo-ablation balloon, has a proximal end fixed to the distal end of the catheter (surrounding the distal end opening), and a distal end fixed to the elongated member, such that rotation of the elongated member relative to the catheter causes the expandable balloon to wrap around the elongated member.

A proximal end of the catheter is preferably coupled to a handle having actuator mechanism, e.g., a slidable knob or thumb ring, mounted thereto, wherein movement of the actuator causes corresponding rotation of the elongated member relative to the catheter. For example, the actuator may be configured for being rotationally displaced relative to the handle and/or axially displaced relative to the handle. The actuator may be directly coupled to the elongated member, or indirectly coupled through a planetary gear system or a set of beveled gears, by way of non-limiting examples. An automatic return mechanism (e.g., a spring) may optionally be associated with the actuator and configured for causing the actuator to return the elongated member to a certain position relative to the catheter in the absence of any external force being applied to the actuator.

The elongated member and catheter body may also be configured for relative axial displacement in addition to (or as an alternative to) rotational displacement, so that the balloon is elongated or compressed by moving the elongated member axially relative to the catheter. For example, the proximal end of the catheter may be fixed to a handle, and the proximal end of the elongated member may be threadedly engaged within the handle, such that rotation of the elongated member relative to the catheter axially displaces the elongated member relative to catheter. In one embodiment, a threaded boss may be mounted to the proximal end of the elongated body, and a threaded collar may be mounted within the handle around the threaded boss. Alternatively, the proximal end of the elongated member may be affixed to the handle, and the proximal end of the catheter may be threadedly engaged within the handle.

The tissue ablation system includes or is otherwise used with a delivery sheath having a lumen through which the catheter is deployed into the patient's heart, with the (deflated) balloon sized and configured for being extended out of, and retracted back into, a distal end opening of the sheath. In use, the catheter is advanced through the delivery sheath lumen until the (deflated) balloon is deployed out of the distal sheath opening, e.g., into a heart chamber. The balloon is then expanded and used to perform the tissue ablation procedure, e.g., ablating one or more target tissue sites in the heart chamber. Thereafter, the balloon is deflated, and the elongated member is rotated relative to the catheter so that the balloon is at least partially wrapped around the elongated member, allowing the balloon to be withdrawn back into the distal opening of the delivery sheath. In some embodiments, the collapsed balloon is elongated axially as an alternative to, or in addition, being wrapped about the elongated member prior to retraction back into the sheath.

Other and further aspects and features of the disclosed embodiments will be evident from reading the following detailed description in view of the accompanying drawings, which are provided for purposes of illustration, and not for purposes of limitation.

### BRIEF DESCRIPTION OF THE DRAWINGS

Referring now to the drawings in which like reference numbers represent corresponding parts throughout and in which:

FIG. 1 is an exploded view of a medical assembly including a balloon folding mechanism, constructed in accordance with the present inventions;

FIGS. 2A and 3A are perspective views of a proximal end of a cryo-ablation apparatus in the neutral and rotated positions, respectively;

FIGS. 2B and 3B are perspective views of a balloon of the cryo-ablation apparatus in the inflated and deflated, folded configurations, respectively;

FIGS. 2C and 3C are end views of the balloon in the inflated and deflated, folded configurations, respectively;

FIGS. 2D and 3D are longitudinal cross-sectional views of the balloon of the cryo-ablation apparatus taken along lines 2D-2D and 3D-3D in FIGS. 2B and 3B, respectively;

FIGS. 4A and 4B are longitudinal cross-sectional views of the proximal end of one embodiment of the cryo-ablation apparatus in the neutral and actuated configurations, respectively;

FIGS. 5A and 5B are longitudinal cross-sectional views of the proximal end of another embodiment of the cryo-ablation apparatus in the neutral and actuated configurations, respectively;

FIG. 6 is a longitudinal cross-sectional view of the proximal end of yet another embodiment of the cryo-ablation apparatus; and

FIGS. 7A-7C are partial cross sectional views of steps in a method of using the medical kit shown in FIG. 1 for performing a medical procedure.

### DETAILED DESCRIPTION OF ILLUSTRATED EMBODIMENTS

Embodiments relate to apparatus and methods for folding a deflated balloon to a smaller profile such that the balloon may easily be retracted into a sheath and safely removed from a patient's body. In this manner, embodiments advantageously rotate, or rotate and elongate, the balloon during or after deflation such that the deflated balloon has a smaller profile than that of conventional devices, which conventional devices may encounter difficulty in retracting the deflated balloon.

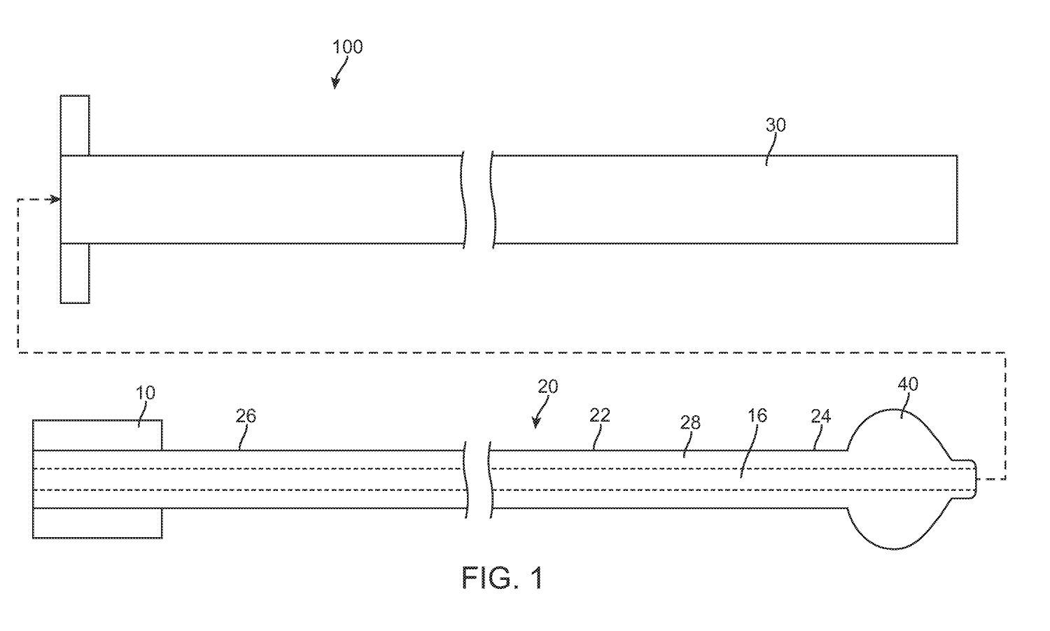

Referring to FIG. 1, an exemplary medical kit 100 constructed in accordance with the present inventions is shown. The medical kit 100 generally includes a catheter 20 and a delivery sheath 30 sized for slidably receiving the catheter 20 therein. In the illustrated embodiment, the catheter 20 is an ablation catheter comprising a catheter body 22, an expandable member 40 attached to the distal end 24 of the catheter body 22, a handle 10 attached to the proximal end 26 of the catheter body 22, and a lumen 28 extending between the proximal end 26 and the distal end 24.

The lumen 28 of the catheter 20 includes an elongated member, or guide wire shaft 16 (shown in phantom in FIG. 1), which may be sized for slidably receiving a guide wire (not shown) or other tool therein. The expandable member 40 may be an expandable balloon for use in a cryogenic ablation procedure. As described in further detail below, the handle 10 includes a balloon folding mechanism that is operated from the proximal end 26 of the catheter 20 to effect balloon folding at the distal end 24 of the catheter 20.

It should be noted that, although the balloon folding mechanisms discussed herein are described as being particularly useful in folding an expandable cryogenic ablation balloon 40, the balloon folding mechanisms can also be used in other balloon catheters where it is desirable to rotate, or rotate and elongate, the balloon prior to retracting the balloon into the sheath 30.

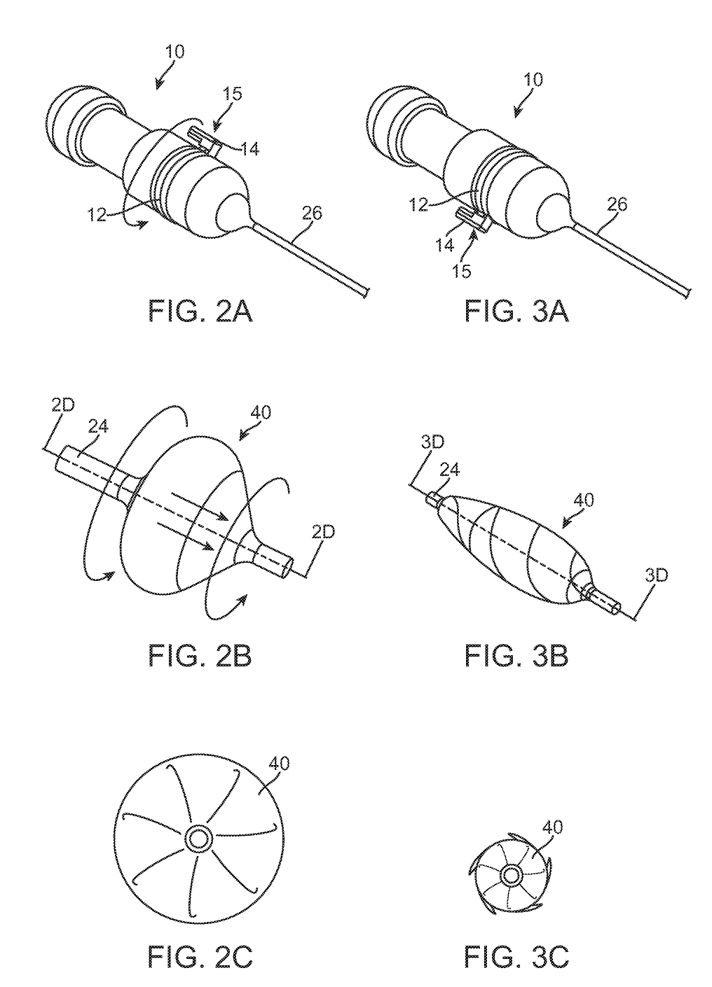

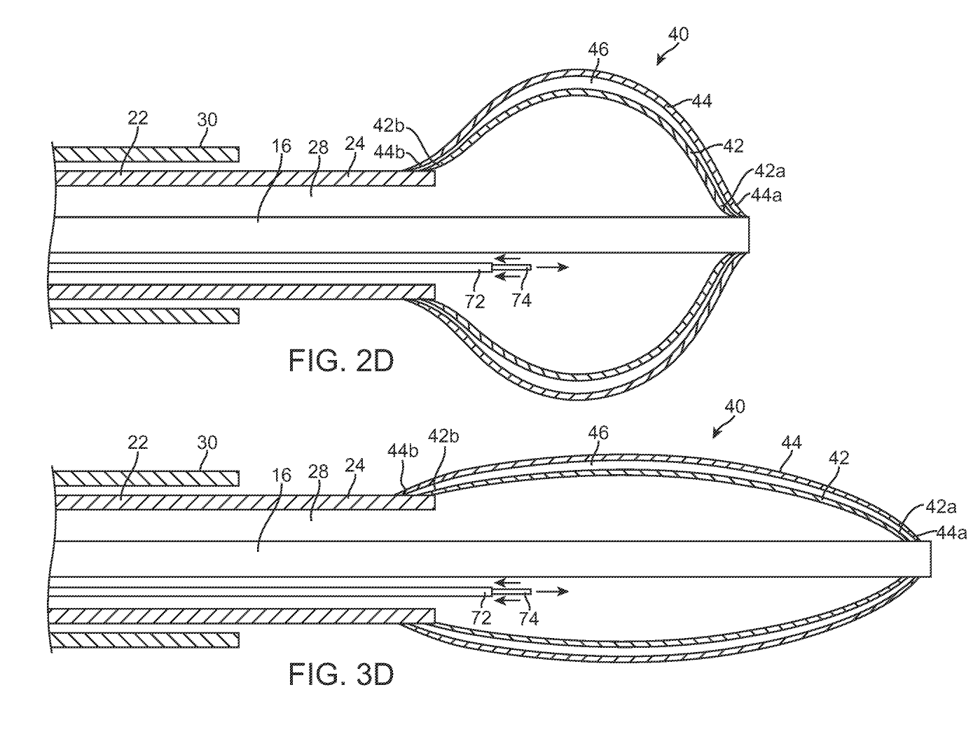

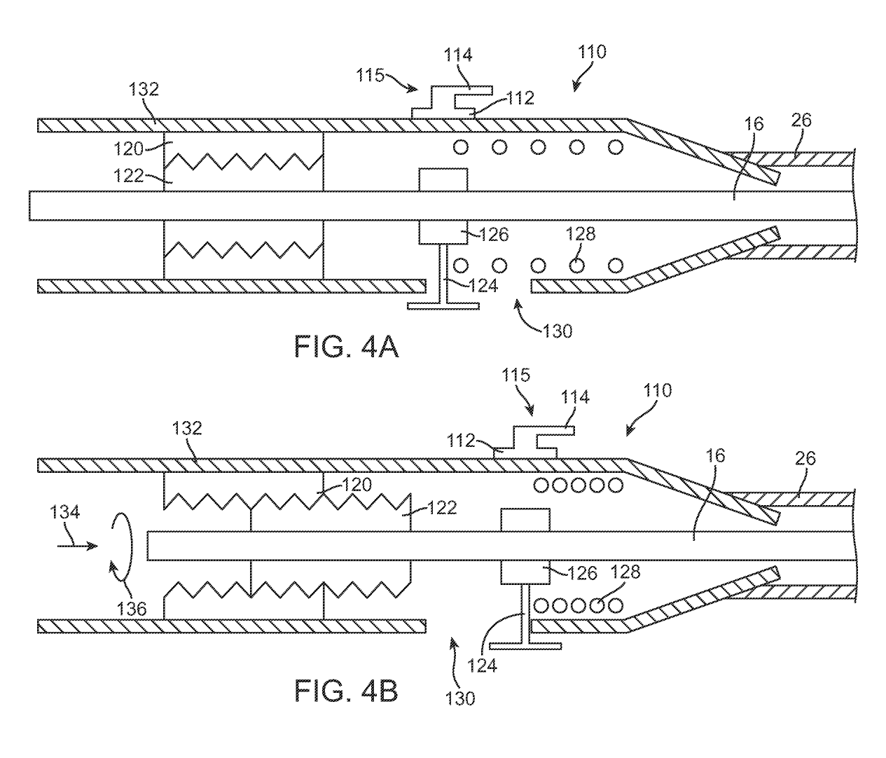

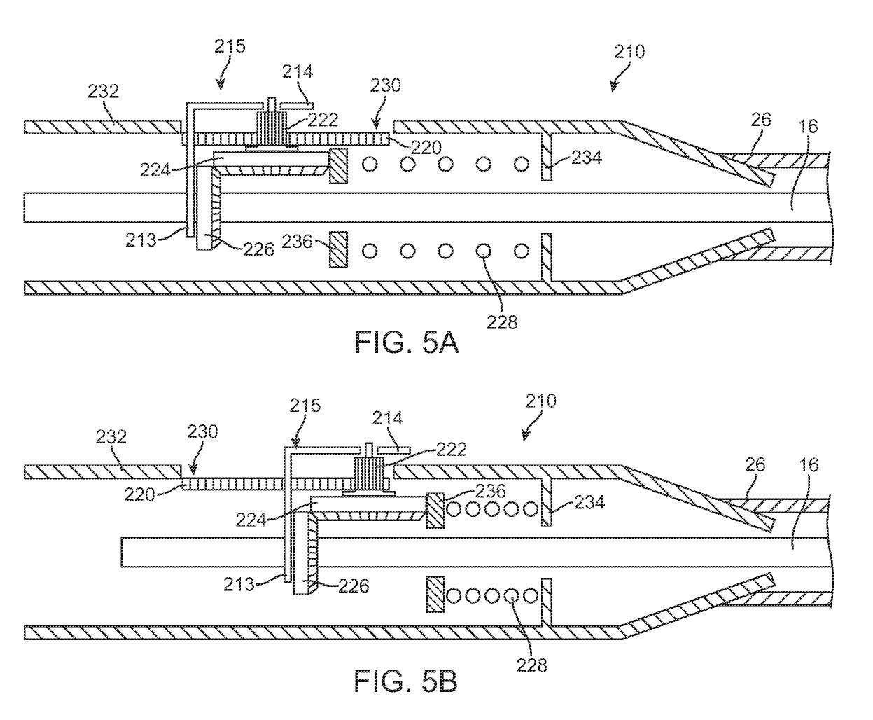

As depicted in FIGS. 2A and 3A, the handle 10 on the proximal end 26 of the catheter 20 may include a rotatable actuator 15. In this example, the actuator 15 takes the form of a thumb ring that includes a rotating member 12 and a tab 14 protruding from the rotating member 12. In this manner, the handle 10 is configured to be operated with one hand with the fingers grasping the handle 10 and the thumb positioned on the protruding tab 14. As will be described in further detail below, the balloon 40 on the distal end 24 of the catheter 20 can be folded by simply pushing the tab 14 with the thumb from the neutral position shown in FIG. 2A to the rotated position shown in FIG. 3A. In order to effect elongation in addition to rotation of the balloon 40, the rotating member 12 may also be configured for axial movement.

The balloon 40 is shown in the inflated state in FIGS. 2B-2D and in the deflated and folded state in FIGS. 3B-3D. In an exemplary embodiment, shown particularly in FIGS. 2D and 3D, the balloon 40 is a dual balloon including an inner balloon wall 42, an outer balloon wall 44, and a space 46 therebetween. The space 46 between the balloon walls 42 and 44 is under vacuum. However, for clarity, the vacuum conduit is not illustrated.

The guide wire shaft 16 extends through the lumen 28 of the catheter body 22, and the distal end of the guide wire shaft 16 extends distally beyond the distal end 24 of the catheter body 22. The distal ends 42a, 44a of the balloon walls 42, 44 are fixedly attached to the distal end of the guide wire shaft 16, and the proximal ends 42b, 44b of the balloon walls 42 and 44 are fixedly attached to the distal end 24 of the catheter body 22. The guide wire shaft 16 is configured to rotate within the catheter lumen 28 relative to the catheter body 22, thereby causing the balloon 40 to wrap around the distal end of the guide wire shaft 16.

The catheter body 22 and the guide wire shaft 16 are laterally flexible, yet torsionally rigid. Torque applied to the proximal end of the guide wire shaft 16 through the balloon folding mechanism in the handle 10 is efficiently translated to the distal end of the guide wire shaft 16 in order to rotate the distal ends 42a, 44a of the balloon walls 42, 44 relative to the proximal ends 42b, 44b of the balloon walls 42, 44. Similarly, the catheter body 22 has enough torsional rigidity to avoid twisting when the distal ends 42a, 44a of the balloon walls 42, 44 are rotated relative to the catheter body 22.

The catheter 20 includes a coolant inlet lumen 74 and an exhaust lumen 72, shown here as concentric lumens, disposed within the lumen 28 of the catheter 20, and configured for transporting liquid coolant between the proximal end of the catheter 20 and the balloon 40 on the distal end of the catheter 20. It should be well understood that the coolant lumen 74 and exhaust lumen 72 may have other configurations and may be arranged for more uniform dispersal of the coolant within the balloon 40.

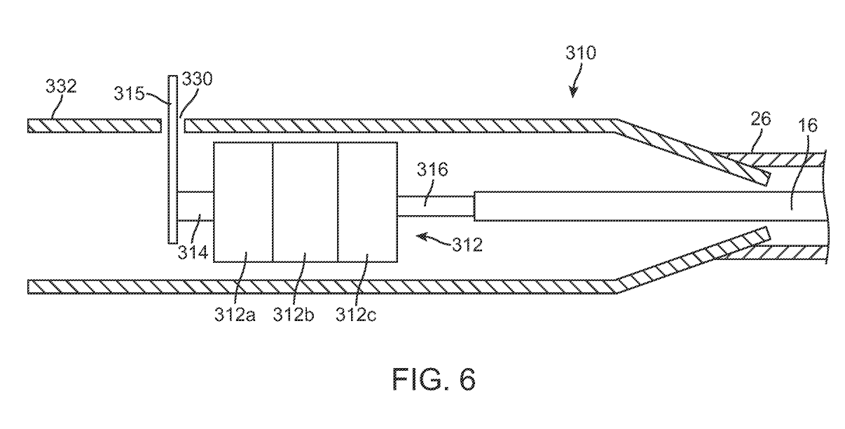

As discussed briefly above, the balloon folding mechanism disposed in the handle may be configured for rotating and elongating the balloon 40. One exemplary embodiment of such a balloon folding mechanism is depicted in FIG. 4A, which shows the folding mechanism in a neutral position, and FIG. 4B, which shows the folding mechanism in the rotated and distally advanced position. A handle 110 attached to the proximal end 26 of the catheter body 22 includes a housing 132 for the balloon folding mechanism components. The proximal end of the guide wire shaft 16 is disposed within the housing 132 and an actuator 115, including a rotating member 112 and a thumb tab 114, is fixedly attached to the guide wire shaft 16 though an attaching member 124 and an annular ring 126.

Although it should be well understood that any mechanism for fixedly coupling the guide wire shaft 16 and the actuator 115 may be employed, in the illustrated embodiment, the attaching member 124 and annular ring 126 form a fixed, direct attachment between the rotating member 112 and the guide wire shaft 16. The attaching member 124 passes through a slot 130 in the housing 132. The slot 130 extends annularly around a portion of the circumference of the housing 132 in order to allow sufficient rotational movement of the attaching member 124 within the slot 130. In addition, the slot 130 is wide enough to allow sufficient axial movement of the attaching member 124 within the slot 130.

The guide wire shaft 16 is fixedly coupled to an externally threaded boss 122 and the housing 132 includes an internally threaded collar 120. Due to the threaded engagement between the boss 122 and the threaded collar 120, rotational movement of the guide wire shaft 16 also causes axial displacement of the guide wire shaft 16 and the rotating member 112. While the axial path of the guide wire shaft 16 is defined by the threaded boss 122 and the threaded collar 120 of the housing 132, the attaching member 124 attached to the rotating member 112 is free to move axially within the slot 130.

The balloon folding mechanism may also include an automatic return mechanism coupled to the rotating member 112. Thus, when the rotating member 112 is rotated and then released, the rotating member 112 will automatically return to the neutral position shown in FIG. 4A. Such an automatic return mechanism may reduce the likelihood of human error in operating the device.

In one embodiment, the automatic return mechanism includes a spring 128 disposed within the housing 132. The distal end of the spring 128 engages an inner wall of the housing 132 and remains stationary. Meanwhile, the proximal end of the spring 128 abuts the attaching member 124, and, thus, moves towards the distal end of spring 128 when the actuator 115 is actuated. In a relaxed configuration, shown in FIG. 4A, the spring 128 biases the actuator 115 to the neutral position.

When the actuator 115 is in the rotated and distally advanced position, shown in FIG. 4B, the spring 128 is compressed. The spring constant is such that the amount of force required to rotate the actuator 115 is sufficient to compress the spring 128. During proximal retraction of the balloon 40 into the sheath 30, a user must maintain the actuator 115 in the rotated and distally advanced position shown in FIG. 4B in order to keep the balloon 40 folded during the retraction. After the balloon 40 is safely retracted and disposed within the sheath 30, the user may release the actuator 115. Upon such release, the spring 128 forces the actuator 115 back to the neutral position shown in FIG. 4A.

During operation of the balloon folding mechanism shown in FIGS. 4A and 4B, the actuator 115 is rotated and distally advanced in order to effect rotation and distal displacement of the guide wire shaft 16. Since the distal ends 42a, 44a of the balloon walls 42, 44 are fixedly coupled to the guide wire shaft 16, as shown in FIGS. 2D and 3D, rotating and distally displacing the actuator 115 therefore also causes the distal ends 42a, 44a of the balloon walls 42, 44 to rotate and distally advance relative to the proximal ends 42b, 44b of the balloon walls 42, 44. Folding the balloon 40 in this manner reduces the profile of the deflated balloon 40, thereby facilitating easier retraction of the balloon 40 proximally into the sheath 30 while avoiding scrunching or entangling the balloon 40.

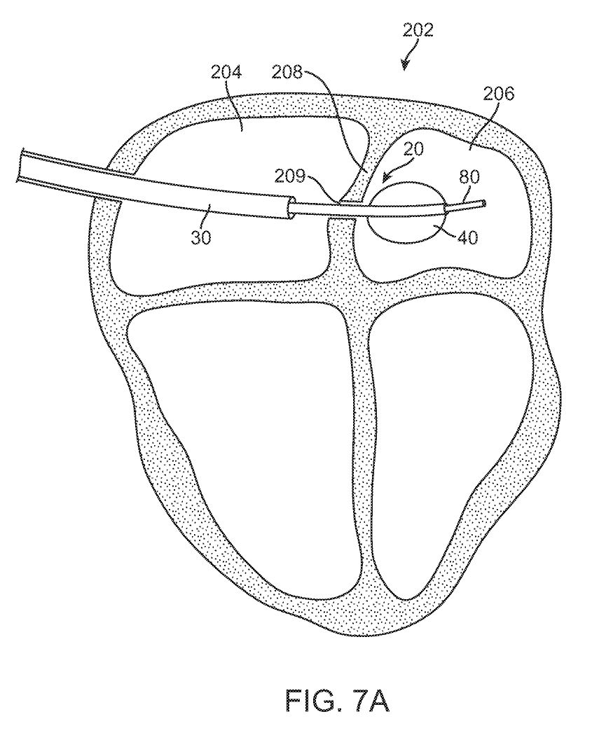

Another exemplary embodiment of a balloon folding mechanism configured for rotating and elongating the balloon 40 is depicted in FIG. 5A, which shows the folding mechanism in a neutral position, and FIG. 5B, which shows the folding mechanism in the rotated and distally advanced position. Similar to the embodiment in FIGS. 4A and 4B, a handle 210 attached to the proximal end 26 of the catheter body 22 includes a housing 232 for the balloon folding mechanism components. The proximal end of the guide wire shaft 16 is disposed within the housing 232 and an actuator 215 is coupled to the guide wire shaft 16 through a spur gear 222 and a set of beveled gears 224 and 226.

In particular, the actuator 215 protrudes from an opening 230 in the housing 232, and includes an external portion 214 configured for being distally advanced, and an inwardly extending portion 213 configured for pushing the vertical beveled gear 226 forward as the actuator 215 is advanced. The spur gear 222 meshes with a static rack gear 220 and is rotatably coupled to the actuator 215 such that the spur gear 222 is distally advanced and rotated along the static rack gear 220 as the actuator 215 is distally advanced. The bottom of the spur gear 222 is fixedly coupled to the horizontal beveled gear 224, such that rotation and axial displacement of the spur gear 222 causes simultaneous rotation and axial displacement of the horizontal beveled gear 224. The horizontal beveled gear 224 meshes with the vertical beveled gear 226 such that horizontal rotation of the horizontal beveled gear 224 causes simultaneous vertical rotation of the vertical beveled gear 226. The vertical beveled gear 226 is fixedly attached to the guide wire shaft 16 such that rotation of the vertical beveled gear 226 causes simultaneous rotation of the guide wire shaft 16.

With this balloon folding mechanism arrangement, rotation and elongation of the balloon 40 is achieved by distally advancing the actuator 215. The ratio between the distal advancement of the actuator 215 and the rotation of the guide wire shaft 16 is dependent upon the gear ratio between the horizontal beveled gear 224 and the vertical beveled gear 226. Thus, the gear ratio can be selected to achieve a desired amount of rotational output at the distal end of the guide wire shaft 16.

Similar to the embodiment shown in FIGS. 4A and 4B, the balloon folding mechanism shown in FIGS. 5A and 5B may include an automatic return mechanism. In particular, a spring 228 disposed between a stationary annular protrusion 234 and a displaceable annular member 236 that abuts the horizontal beveled gear 224 may serve as the automatic return mechanism. As shown in FIG. 5A, when the balloon folding mechanism is in a neutral position, the spring 228 is in an extended, neutral configuration.

As shown in FIG. 5B, distal advancement of the actuator 215 causes the annular member 236 to move towards the annular protrusion 234, thereby compressing the spring 228. The spring constant is such that the amount of force required to distally advance the actuator 215 is sufficient to compress the spring 228. During proximal retraction of the balloon 40 into the sheath 30, a user must maintain the actuator 215 in the distally advanced position shown in FIG. 5B in order to keep the balloon 40 folded during the retraction. After the balloon 40 is safely retracted and disposed within the sheath 30, the user may release the actuator 215. Upon such release, the spring 228 forces the actuator 215 back to the neutral position shown in FIG. 5A.

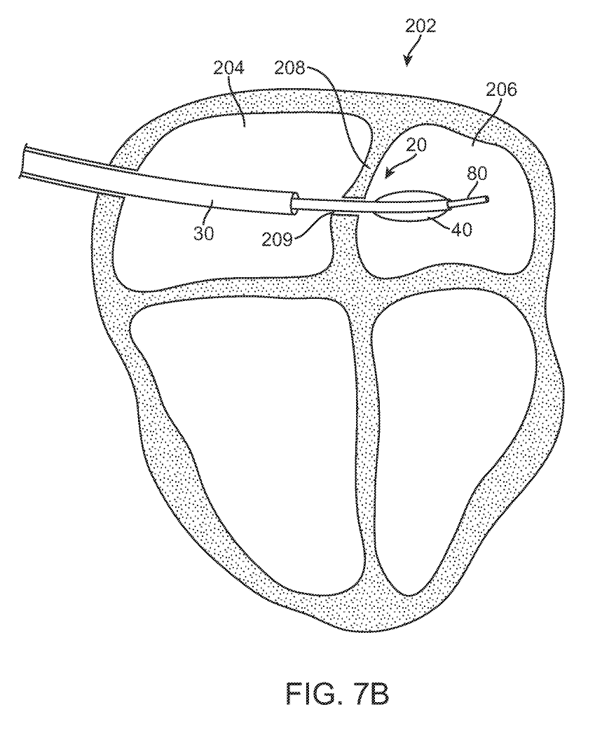

An exemplary embodiment of a balloon folding mechanism configured for rotating the balloon 40 is depicted in FIG. 6. Similar to the embodiments shown in FIGS. 4A-5B, a handle 310 attached to the proximal end 26 of the catheter body 22 includes a housing 332 for the balloon folding mechanism components. The proximal end of the guide wire shaft 16 is disposed within the housing 332 and is coupled to an actuator 315 through an input shaft 314, a planetary gear system 312 and an output shaft 316. In particular, the actuator 315 protrudes from an opening 330 in the housing 332 and is configured for being rotated by the user. Thus, the opening 330 extends circumferentially around a portion of the housing 332. The actuator 315 is coupled to the planetary gear system 312 through the input shaft 314. The output shaft 316 of the planetary gear system 312 is coupled (i.e., by bonding, gluing, or the like) to the guide wire shaft 16.

Due to the overall gear ratio in the planetary gear system 312, a small amount of rotation of the actuator 315 results in a large amount of rotation of the guide wire shaft 16. For example, the ratio of input rotation of the actuator 315 to output rotation of the guide wire shaft 16 may be between 1:4 and 1:400. That is, one degree of rotation of the actuator 315 may produce between 4 and 400 degrees of rotation of the guide wire shaft 16. Because such a large amount of rotational output is sufficient for achieving a reduced profile of the deflated balloon 40, the balloon folding mechanism illustrated in FIG. 6 does not require elongation of the balloon 40.

In the illustrated embodiment, there are three sets of planetary gears 312a, 312b, 312c in the planetary gear system 312. The sets of planetary gears 312a, 312b, 312c are conventional planetary gear sets that each include a center sun gear, planet gears meshed with the sun gear, a planet gear carrier, and an outer annulus with inward-facing teeth that mesh with the planet gears. The gear sets 312a, 312b, 312c are arranged in series such that a common longitudinal axis passes through the center of each of the sun gears. In this manner, the output of the first gear set 312a is coupled to the input of the second gear set 312b, the output of the second gear set 312b is coupled to the input of the third gear set 312c, and the output of the third gear set 312c is coupled to the output shaft 316.

Any one of the components of each of the gear sets 312a, 312b, 312c may be chosen as the input and any one of the other components may be chose as the output. In one example, the annulus of each of the gear sets 312a, 312b, 312c remains stationary, the planet gear carrier is the input of each set, and the sun gear is the output of each set. In particular, the actuator 315 is coupled, through the input shaft 314, to the planet gear carrier of the first gear set 312a, the sun gears of the first and second gear sets 312a, 312b are coupled to the planet gear carriers of the second and third gear sets 312b, 312c, respectively, and the sun gear of the third gear set 312c is coupled to the output shaft 316. However, it should be well understood by one of ordinary skill in the art that any planetary gear system arrangement could be used to achieve the desired output rotation. For example, there may be more or less than three sets of planetary gears, the ratio of input rotation to output rotation can be chosen to achieve a desired folded profile of the deflated balloon, the input component of each gear set could alternatively be the sun gear or the annulus, and the output component of each gear set could alternatively be the planet gear carrier or the annulus.

It should be understood that, for the sake of clarity, several elements of the handles 110, 210 and 310 are not shown in FIGS. 4A-6. For example, the handles 110, 210 and 310 may include a vacuum port, a vacuum lumen, a coolant inlet port, a coolant inlet lumen, a coolant outlet port, a coolant outlet lumen, and the like.

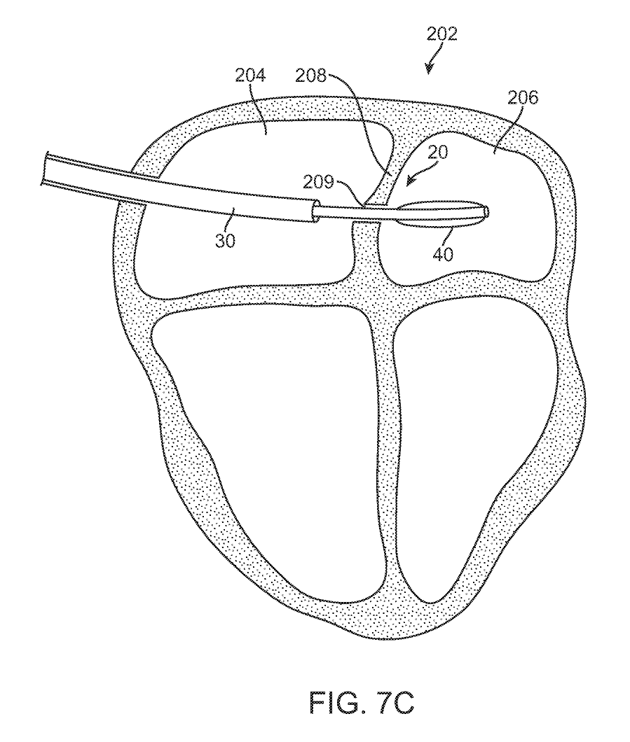

Having described the structure and operation of different embodiments of the balloon folding mechanism, the operation of the medical kit assembly 100 in performing an exemplary therapeutic ablation procedure within a left atrium will now be described with reference to FIGS. 7A-7C. Although the method of using the kit 100 is depicted as taking place in the left atrium, the kit 100 is not restricted to use within the left atrium and may advantageously be used in other areas of the body.

First, with reference to FIG. 7A, the catheter delivery sheath 30 is introduced into the right atrium 204 of the heart 202 via the appropriate blood vessel. The catheter 20 is advanced through the sheath 30 while the sheath 30 remains within the right atrium 204. A guide wire 80 is inserted through the guide wire shaft 16 (not shown in FIG. 7A) of the catheter 20 and advanced, such that the distal end of the guidewire 80 is located at a target site within the left atrium 206 (e.g., a pulmonary vein). The catheter 20 is then further advanced from the right atrium 204 along the guidewire 80 into the left atrium 206 by passing through an opening 209 in the atrial septum 208. Once the catheter 20 is properly positioned within the left atrium 206, liquid coolant flows into the balloon 40 through the coolant inlet lumen 74 (see FIGS. 2D and 3D) to inflate the balloon 40 from its original geometry to an expanded geometry, as shown in FIG. 7A, and an ablation procedure is performed in a conventional manner.

Although the illustrated embodiment depicts a transeptal approach for entering the left atrium 206, it should be well understood that a conventional retrograde approach, i.e., through the respective aortic and mitral valves of the heart, may alternatively be used for entering the left atrium 206. In addition, it should be well understood that, although the illustrated embodiment depicts the catheter 20 passing through the atrial septum 208, the sheath 30 may also traverse the atrial septum 208 in the method of using the medical kit 100.

After the ablation procedure is completed and the liquid coolant is discharged from the balloon 40 through the discharge lumen 72 (see FIGS. 2D and 3D), the balloon 40 is deflated (e.g., using vacuum or other conventional deflation procedures) to a collapsed geometry, shown in FIG. 7B, which has a slightly larger profile than the original geometry of the balloon 40 prior to inflation. If the deflated balloon 40 has an outer diameter that is larger than the inner diameter of the sheath 30 and/or larger than the opening 209 in the septum 208, it may be difficult to withdraw the balloon 40 back into the sheath 30 and/or back through the opening 209 without tearing or otherwise damaging the balloon 40 and/or the atrial septum 208.

Thus, one of the balloon folding mechanisms described above may advantageously be employed to minimize the profile of the deflated balloon 40, thereby facilitating withdrawal of the balloon 40 back into the sheath 30 and/or back through the atrial septum 208. Folding the balloon 40 may include rotating the balloon 40 (i.e., using the folding mechanism depicted in FIG. 6) and may additionally include elongating the balloon 40 (i.e., using one of the balloon folding mechanisms depicted in FIGS. 4A-5B), depending on which folding mechanism is employed.

As depicted in FIG. 7C, actuating the balloon folding mechanism folds the deflated balloon 40 into a profile that is smaller than the collapsed geometry shown in FIG. 7B. Actuating the balloon folding mechanism may include both rotating and distally advancing an actuator (i.e., the actuator 115 shown in FIGS. 4A and 4B), only distally advancing an actuator (i.e., the actuator 215 shown in FIGS. 5A and 5B), or only rotating an actuator (i.e., the actuator 315 shown in FIG. 6). Due to the smaller profile of the folded balloon 40, retracting the balloon 40 into the sheath 30 and/or back through the opening 209 in the atrial septum 208 is easier. When the balloon 40 is safely positioned within the sheath 30, the user may release the actuator of the balloon folding mechanism, and the sheath 30 with the balloon 40 therein may then be safely removed from the patient.

Although particular embodiments have been shown and described, it should be understood that the above discussion is not intended to limit the scope of these embodiments. Various changes and modifications may be made without departing from the scope of the claims. For example, the catheter may include types of balloons other than cryo-ablation balloons. Further, the balloon folding mechanism may be configured for rotating the catheter body 22 about the guide wire shaft 16 (rather than the guide wire shaft 16 being rotated within the catheter body 22). Further, in the embodiment illustrated in FIGS. 4A and 4B, the threaded engagement may be between an inner surface of the rotating member 112 and an outer surface of the housing 132, rather than between the guide wire shaft 16 and the inner surface of the housing 132. Further, in the embodiment illustrated in FIGS. 4A and 4B, the threads may be removed and the rotating member 112 may be configured for independent axial and rotational movement.

Thus, embodiments are intended to cover alternatives, modifications, and equivalents that may fall within the scope of the claims.

What is claimed is:

1. A catheter assembly, comprising:
a catheter body;
a handle coupled to the catheter body;
an elongated member extending through a lumen of the catheter body, with a distal end of the elongated member extending out of an opening in a distal end of the catheter body in communication with the lumen;
an expandable balloon having a proximal end secured to the distal end of the catheter body surrounding the distal opening therein, and a distal end secured to the distal end of the elongated member, one of the elongated member and catheter body being rotatable relative to the other, such that the balloon, when deflated, can be at least partially wrapped around a distal end portion of the elongated member by such relative rotation, and
an actuator mounted on the handle and coupled to one of the catheter body and elongated member, wherein movement of the actuator causes rotation of the elongated member and catheter body relative to each other.

2. The catheter assembly of claim 1, wherein the handle is affixed to a proximal end of the catheter body, and the actuator is coupled to a proximal end of the elongated member, such that actuating the actuator causes the elongated member to rotate within the lumen of the catheter body.

3. The catheter assembly of claim 1, wherein the actuator is configured for being rotationally displaced relative to the handle.

4. The catheter assembly of claim 1, wherein the actuator is configured for being axially displaced relative to the handle.

5. The catheter assembly of claim 1, wherein the elongated member is a guide wire shaft.

6. The catheter of assembly of claim 1, wherein the elongated member and catheter body are configured for being axially displaced relative to each other, such that the balloon, when deflated, can be elongated by such relative axial motion.

7. The catheter assembly of claim 6, wherein one of the elongated member and the catheter body is affixed to the handle, and the other of the elongated member and the catheter body is threadedly engaged within the handle, such that rotation of the elongated member and catheter body relative to each other axially displaces the elongated member and catheter body relative to each other.

8. The catheter assembly of claim 7, wherein a proximal end of the catheter body is affixed to the handle, and a proximal end of the elongated body is threadedly engaged with the handle.

9. The catheter assembly of claim 8, further comprising a threaded boss mounted to the proximal end of the elongated body, and a threaded collar mounted within the handle around the threaded boss.

10. The catheter assembly of claim 1, further comprising an automatic return mechanism associated with the actuator, the return mechanism configured for forcing the actuator to return to a preset position when an external force is released from the actuator.

11. The catheter assembly of claim 10, the automatic return mechanism comprising a spring positioned between the actuator and an inner surface of the handle.

12. The catheter assembly of claim 1, wherein the balloon is a cryo-ablation balloon.

13. The catheter assembly of claim 1, wherein the actuator is coupled to the elongated member through a planetary gear system.

14. The catheter of claim 1, wherein the actuator is coupled to the elongated member through a set of beveled gears.

15. A medical assembly, comprising:
the catheter assembly of claim 1; and
a delivery sheath having a lumen through which the catheter body may be extended, the sheath being sized relative to the catheter body so that the balloon may be deployed out of a distal opening in the sheath in communication the sheath lumen.

16. A method for performing a medical procedure on a patient using the medical assembly of claim 15, comprising:
advancing the catheter body through delivery sheath lumen until the expandable balloon is deployed through the distal opening in the sheath;
expanding the balloon;
performing the medical procedure on the patient using the expanded balloon;
at least partially collapsing the expandable balloon after performing the medical procedure;
rotating the elongated member and the catheter body relative to each other, such that the at least partially collapsed balloon at least partially wraps around the elongated member; and
retracting the catheter body and balloon back through the distal sheath opening into the delivery sheath lumen.

17. The method of claim 16, wherein the performing the medical procedure comprises ablating a target tissue site.

18. The method of claim 16, further comprising elongating the at least partially collapsed balloon by axial extension of the elongated body relative to the catheter body.

19. The method of claim 16, wherein rotating the elongated member and the catheter body relative to each other comprises rotating the actuator relative to the handle.

20. The method of claim 16, wherein rotating the elongated member and the catheter body relative to each other comprises distally advancing the actuator relative to the handle.
